(19) United States
(12) Patent Application Publication
(10) Pub. No.: US 2005/0209661 A1
(43) Pub. Date: Sep. 22, 2005

(54) System and method for generating electromagnetic fields of varying shape based on a desired target

(75) Inventor: Reza Kassayan, Redwood City, CA (US)

Correspondence Address: SQUIRE, SANDERS & DEMPSEY L.L.P, 600 HANSEN WAY, PALO ALTO, CA, 94304-1043, US

(73) Assignee: Solatronix, Inc., Stanford, CA (US)

(21) Appl. No.: 10/806,486

(22) Filed: Mar. 22, 2004

(51) Int. Cl. A61F 2/00

(52) U.S. Cl. 607/101

(57) ABSTRACT

A system for ablating tissue using interferential electromagnetic fields, comprises tumor shape information; radiation tip shape and position information; a mathematical model for computing first frequency and phase information, second frequency and phase information, and mixing information based on the tumor shape information and on the radiation tip shape and position information. The system further comprises a first generator mechanism for generating a first tone based on the first frequency information and on the first phase information; a second generator mechanism for generating a second tone based on the second frequency information and on the second phase information; a mixer for mixing the first and second tones based on the mixing information; and a radiation tip for generating an interferential electromagnetic field pattern based on the first and second tones. The radiation tip may include radiation coils affixed to the distal end of a conduction member. The conduction member may include a set of nesting conductors for transmitting current to the radiation coils within the radiation tip, the current being selected based on the interferential electromagnetic field pattern desired.

### BACKGROUND OF THE INVENTION

1. Field of the Invention

This invention relates generally to electromagnetic fields, and more particularly provides a system and method for interferentially varying the shape and intensity of electromagnetic fields to a desired target.

2. Description of the Background Art

The heating or ablation of tissue has been a proven effective treatment of several medical problems. For example, menorrhasia is a common condition that inflicts women over the age of forty, and manifests itself as excessive bleeding from the endometrium (the inner wall of the uterus). Menorrhasia can be alleviated and/or cured by wholly or partially destroying the endometrium, for example, by heating the tissue to a temperature of around 60 degrees Celsius for a period of up to five minutes. An example prior art tool for heating endometrial tissue is a probe that generates an electromagnetic field, and is illustrated in FIG. 1 and described in U.S. Pat. No. 6,635,055. The probe of FIG. 1 emanates a heat pattern of a fixed shape, customized for the shape of the endometrium. The shape cannot be modified without modifying the device itself. The probe is therefore a poor choice for other applications.

Ablation therapy can be used for the treatment of tumors. Prior art tumor ablation systems apply an electric current to ablate the tissue of the tumor. To generate electric current through a tumor, a first electrode is placed at the tumor site and a second electrode is placed typically on the hip. Electric current is applied to the tumor electrode. The electric current travels through the body to the hip electrode. Systems like this have been approved by the FDA, e.g., those developed by RITA Medical Systems, Le Veen (Boston Scientific Multiple Tines) and Radionics (a division of Tyco). However, these systems can damage healthy tissue in regions between the tumor electrode and the hip electrode. Also, the skin around the hip electrode often burns.

The probe of these ablation systems is designed to propagate microwave electromagnetic energy in a radial direction from a single focal point, thereby generating a substantially spherical radiation pattern expanding outwards gradually as time passes. Since the electric field is heated from a single focal point, a temperature gradient is created. Temperature effectively decreases as distance from the focal point increases. Accordingly, to heat tissue a distance away from the focal point, the focal point itself must become quite hot. To reduce these unwanted effects, physicians can use lower power settings for longer periods of time, can use multiple probes, or can reapply the same probe in various positions. However, maneuvering multiple tools and tolerating longer procedures become difficult for the physician and for the patient.

The strength and duration of the electric field applied affect the temperature and speed of the ablation results. Generally, at 42 degrees Celsius, a cell dies after approximately 60 minutes. At temperatures between 42-45 degrees Celsius, cells are more susceptible to damage by other agents. At temperatures between 50-52 degrees Celsius, cellular death typically occurs in 4-6 minutes. If, however, heat is applied too quickly to tissue, tissue vaporization may occur. Without a pathway to allow the vapor generated to be released from the body, the vapor may travel to unwanted regions causing medical problems.

Accordingly, a system and method are needed that can heat tissue quickly in a pattern based on the size and shape of a target and without causing unwanted tissue vaporization.

### SUMMARY

Some embodiments described herein are understood to be particularly useful for the treatment of tumors deemed not highly malignant. Some embodiments are thought to be particular useful for the treatment of liver and breast cancer. Some embodiments herein function to heat tissue to 50 to 100 degrees Celsius for four to six minutes without causing charring or vaporization. Some embodiments described herein are intended for use with any ultrasonic system currently available in many hospitals today.

An embodiment of the present invention provides a probe for generating electromagnetic fields of varying shapes and intensities. The size and shape of the electromagnetic fields are based on interferential waves radiating from one or more radiation coils disposed at the head of the probe. The probe may be used to ablate tissue and may be implemented within a medical system that assists a physician with the positioning of the probe and with the generation of an electric field pattern related to the target tissue size and shape to be ablated.

Another embodiment of the present invention provides a probe for generating an electromagnetic field. The probe comprises a conduction member for conducting at least two current signals; and a radiation tip coupled to the conduction member for radiating an electromagnetic field based on the at least two current signals.

Another embodiment of the present invention provides a method for generating an electromagnetic field. The method comprises conducting at least two current signals; and radiating an electromagnetic field based on the at least two current signals.

Another embodiment of the present invention provides a probe for generating an electromagnetic field. This probe comprises a first conductor for receiving a first electric current signal; a second conductor for receiving a second electric current signal; a first radiation coil coupled to the first conductor for radiating a first electromagnetic field based on the first electric current signal; and a second radiation coil coupled to the first conductor for radiating a second electromagnetic field based on the second electric current signal, the first and second electromagnetic fields causing an interferential electromagnetic field pattern.

Another embodiment of the present invention provides a method for generating an electromagnetic field. The method comprises receiving a first current signal; receiving a second current signal; radiating a first electromagnetic field based on the first current signal; and radiating a second electromagnetic field based on the second current signal, the first and second electromagnetic fields causing an interferential electromagnetic field pattern.

Yet another embodiment of the present invention provides a tissue ablation system for ablating tissue using interferential electromagnetic fields. The system comprises tumor shape information; radiation tip shape and position information; a mathematical model for computing first frequency and phase information, second frequency and phase information, and mixing information based on the tumor shape information and on the radiation tip shape and position information; a first generator mechanism for generating a first tone based on the first frequency information and on the first phase information; a second generator mechanism for generating a second tone based on the second frequency information and on the second phase information; a mixer for mixing the first and second tones based on the mixing information; and a radiation tip for generating an interferential electromagnetic field pattern based on the first and second tones.

Still another embodiment of the present invention provides a probe that comprises a radiation tip a conduction member; and a conduction member coupled to the radiation tip and having a circulation region for circulating coolant and having a shield for substantially preventing the coolant from entering the radiation tip.

Another embodiment of the present invention provides a radiation tip for generating an electromagnetic field pattern to ablate tissue. The radiation tip comprises a first radiation coil for radiating a first electromagnetic field based on a first current signal; and a second radiation coil for radiating a second electromagnetic field based on a second current signal, the first electromagnetic field and the second electromagnetic field causing an interferential electromagnetic field pattern for ablating tissue.

Another embodiment of the present invention provides a method for generating an electromagnetic field pattern to ablate tissue. A method comprises radiating a first electromagnetic field based on a first current signal; and radiating a second electromagnetic field based on a second current signal, the first electromagnetic field and the second electromagnetic field causing an interferential electromagnetic field pattern for ablating tissue.

Yet another embodiment of the present invention provides a multiple phase generator. The multiple phase generator comprises a ferromagnetic core; a primary input encircling the ferromagnetic core for supplying a primary wave having a primary frequency and a primary phase to the ferromagnetic core; and at least one pickup encircling and rotatable about the ferromagnetic core for receiving the primary wave, and for generating an output wave having an output frequency substantially equal to the primary frequency and an output phase based on the angle of rotation relative to the primary input.

### BRIEF DESCRIPTION OF THE DRAWINGS

FIG. 1 is a cross-sectional view of a prior art ablation probe.

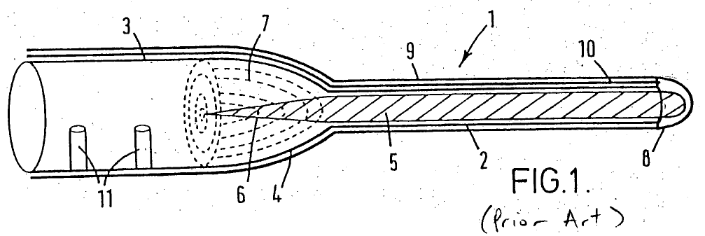

FIG. 2 is a cross-sectional view of a tumor and best matching elliptical ablation pattern in accordance with an embodiment of the present invention.

FIG. 3AB is a perspective view of a first portion of a probe in accordance with an embodiment of the present invention.

FIG. 3CD is a perspective view of a second portion of the probe of FIG. 3AB.

FIG. 3EF is a perspective view of a third portion of the probe of FIG. 3AB.

FIG. 4 is a top view of the radiation coils of the probe in accordance with an embodiment of the present invention.

FIG. 5 is a contour graph showing a first electromagnetic field intensity pattern at the outer boundary of the probe with the application of two tones.

FIG. 6 is a second contour graph showing a second electromagnetic field intensity pattern at the outer boundary of the probe with the application of two tones.

FIG. 7 is a block diagram illustrating an interferential electromagnetic field generation system in accordance with an embodiment of the present invention.

FIG. 8 is a diagram illustrating details of a multiple phase generator for the system of FIG. 7.

FIG. 9 is a block diagram illustrating a computer system in accordance with an embodiment of the present invention.

FIG. 10A is a block diagram illustrating the computer control in accordance with an embodiment of the present invention.

FIG. 10B is a flowchart illustrating a method of controlling the computer control components of FIG. 10A of the computer system of FIG. 9 to generate an electric field based on a target.

FIG. 11 is a diagram illustrating a user interface for receiving physician input and representing diagrammatically the radiation pattern to be generated by the interferential electromagnetic field generation system of FIG. 7.

### DETAILED DESCRIPTION

The following description is provided to enable any person skilled in the art to make and use the invention, and is provided in the context of a particular application and its requirements. Various modifications to the embodiments will be readily apparent to those skilled in the art, and the generic principles defined herein may be applied to other embodiments and applications without departing from the spirit and scope of the invention. Thus, the present invention is not intended to be limited to the embodiments shown, but is to be accorded the widest scope consistent with the principles, features and teachings disclosed herein.

Some embodiments described herein are understood to be particularly useful for the treatment of tumors deemed not highly malignant. Some embodiments are thought to be particular useful for the treatment of liver and breast cancer. Some embodiments herein function to heat tissue to 50 to 100 degrees Celsius for four to six minutes without causing charring or vaporization. Some embodiments described herein are intended for use with any ultrasonic system currently available in many hospitals today.

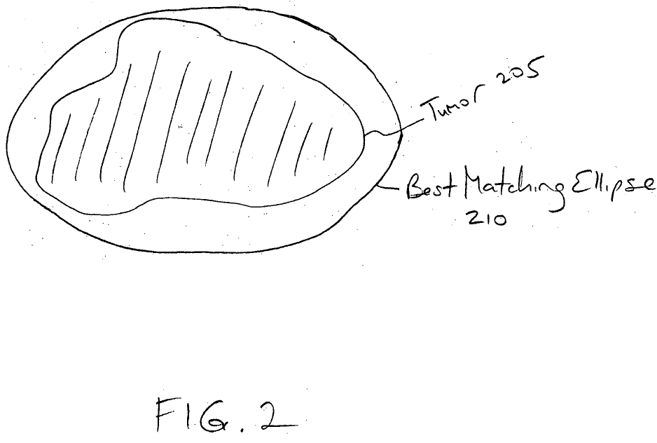

FIG. 2 is a cross-sectional view of a tumor 205 and best matching elliptical ablation pattern 210, in accordance with an embodiment of the present invention. As can be seen in the figure, the tumor 205 is not circular and does not have a smooth surface pattern. Accordingly, a circular (i.e., spherical) ablation pattern would damage a large amount of healthy tissue. Accordingly, a more appropriate ablation pattern may be selected as elliptical pattern 210. Using the system of the present invention, an elliptically shaped electromagnetic field can be applied from almost any point at or near tumor 205. Alternative ablation patterns are also possible.

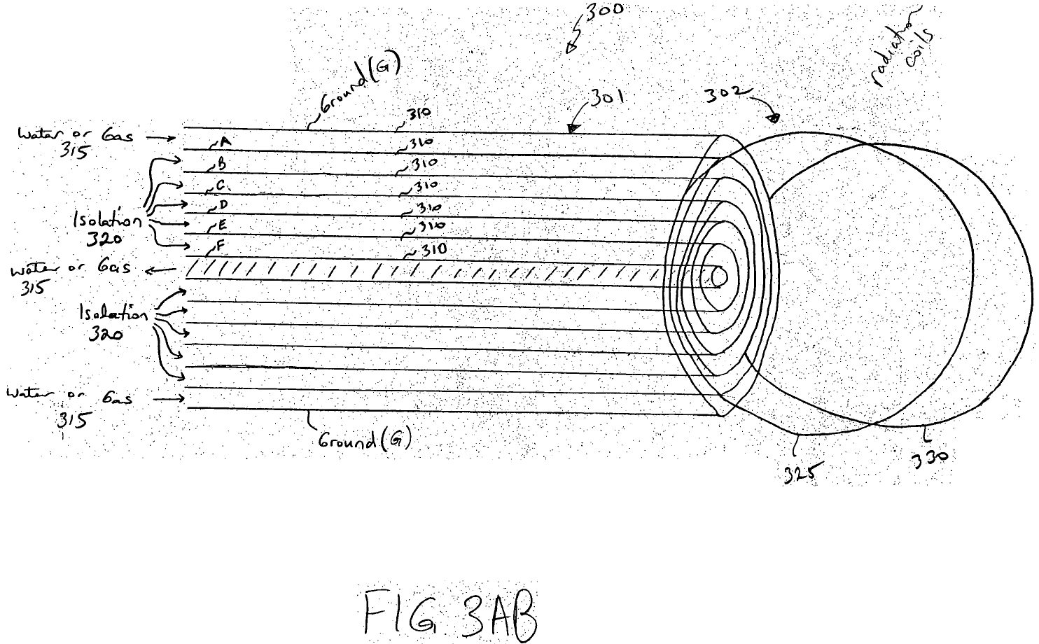

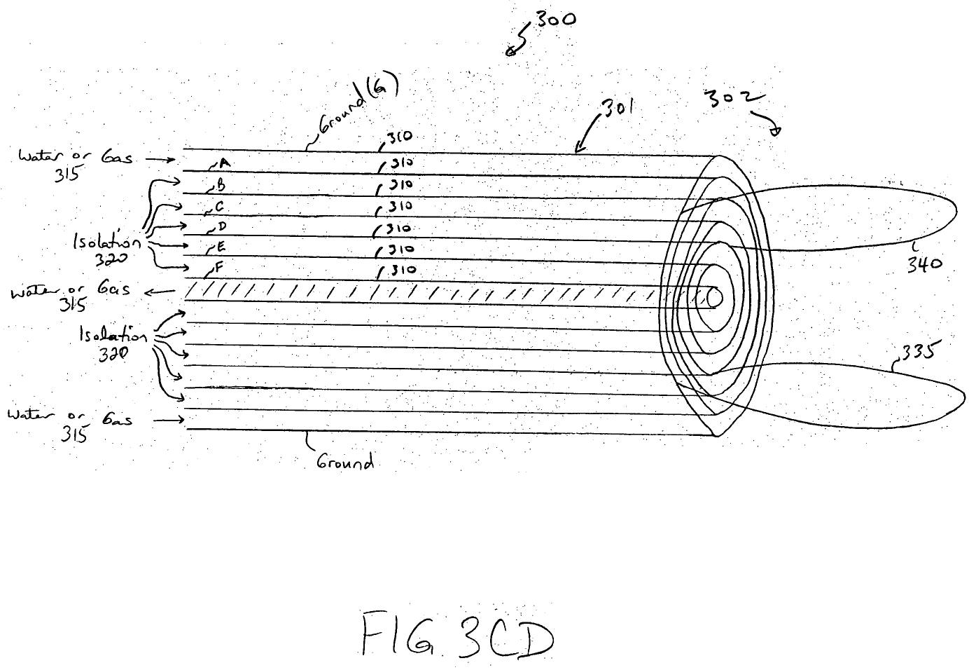

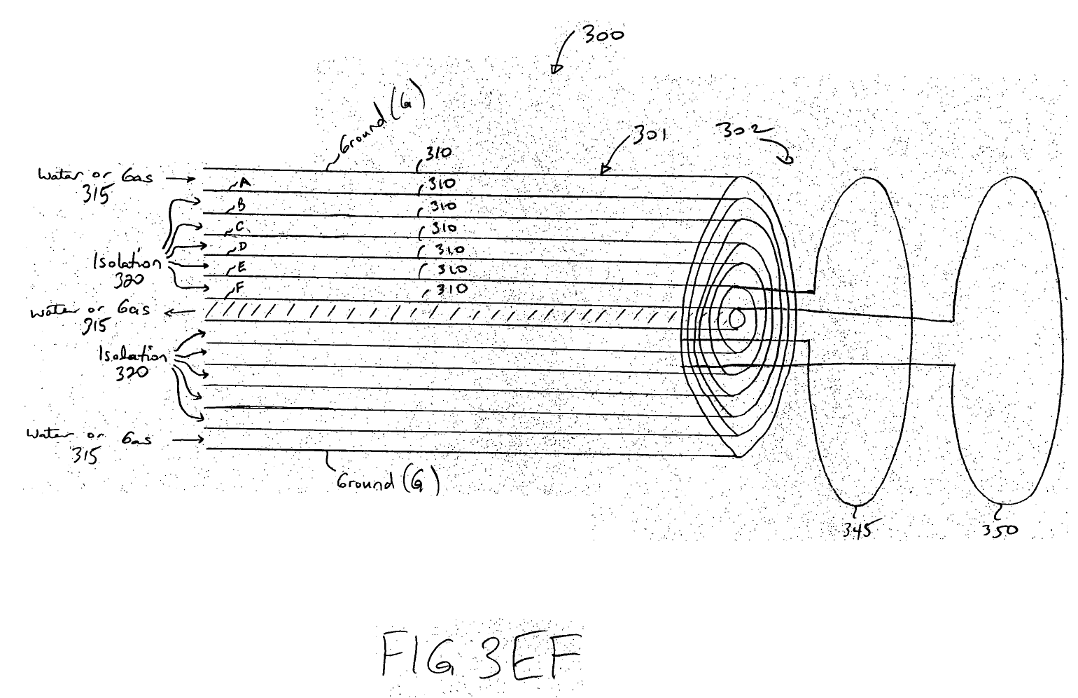

In accordance with an embodiment of the present invention, FIGS. 3AB, 3CD and 3EF show a probe 300 for generating an electromagnetic field pattern of varying shape and intensity. The size and shape of the electromagnetic field are based on interferential waves radiating from one or more radiation coils disposed at the head of the probe 300. Different portions of the single probe embodiment 300 are shown in the three figures for convenience and clarity. The probe 300 may be between 10 and 20 centimeters in length and between 3 and 10 millimeters in diameter, preferably around 4-7 millimeters in diameter. One skilled in the art will recognize that different lengths and diameters can be used based on the intended use of the probe 300.

The probe 300 includes a conduction member 301 and a radiation tip 302 coupled to the distal end of the conduction member 301. In this embodiment, conduction member 301 includes a set of nesting coaxial conductors 310. One skilled in the art will recognize that the conductors 310 need not be disposed coaxially in conduction member 301. Each conductor 310 may be made of any conductive material such as copper, may be substantially cylindrical in length, and may be about 10 centimeters long. Although shown as nesting at substantially equal distances from one another, one skilled in the art will recognize that the conductors 310 can be spaced apart at different distances. In the illustrated example, the conduction member 301 includes seven (7) nesting conductors 310, namely, conductors A-G. Conductor F nests within conductor E, which nests within conductor D, which nests within conductor C, which nests within conductor B, which nests within conductor A, which nests within conductor G.

The outermost conductor 310, namely, conductor G, is preferably coupled to ground. Each of the remaining conductors 310, namely, conductors A-F, receive a predetermined electric current signal that is transmitted from the proximal end of the conduction member 301 (the end opposite the distal end coupling the radiation tip 302) through the particular conductor 310 to the radiation tip 302. The predetermined electric current signal through each of the conductors 310 has a preselected frequency, phase and intensity, so that the electromagnetic fields radiating from the radiation tip 302 and caused by the multiple electric current signals across the conductors 310 form a desired interferential pattern. One skilled in the art will recognize that each current signal applied to a single conductor 310 may be formed from multiple currents of different frequencies, phases and/or intensities to cause an interferential electric current, which causes an interferential wave pattern from a single radiation coil (discussed below) of the radiation tip 302.

Radiation tip 302 includes one or more radiation coils for radiating an electromagnetic filed pattern based on the current received from the conductors 310. In the embodiment shown in FIG. 3, radiation tip 302 includes six (6) radiation coils 325, 330, 335, 340, 345 and 350, each coil placed in a predetermined geographic position of radiation tip 302 and running a substantially circular path (although one skilled in the art will recognize that other shape paths are also possible). FIG. 3AB illustrates details of the first and second radiation coils 325 and 330. FIG. 3CD illustrates details of the third and fourth radiation coils 335 and 340. FIG. 3EF illustrates details of the fifth and sixth radiation coils 345 and 350. Each radiation coil 325-350 is coupled from a respective one of conductors A-F to common ground conductor G. More specifically, radiation coil 325 is coupled between conductor A and conductor G, and forms a somewhat circular path along an east plane of radiation tip 302. Radiation coil 330 is coupled between conductor B and conductor G, and forms a somewhat circular path along a west plane of radiation tip 302. Radiation coil 335 is coupled between conductor C and conductor G, and forms a somewhat circular path along a south plane of radiation tip 302. Radiation coil 340 is coupled between conductor D and conductor G, and forms a somewhat circular path along a north plane of radiation tip 302. Radiation coil 345 is coupled between conductor E and conductor G, and forms a somewhat circular path along a plane perpendicular to each of the east, west, north and south planes and near the bottom of radiation tip 302 (the portion of the radiation tip 302 that is closer to the distal end of the conduction member 301). Lastly, radiation coil 350 is coupled between conductor F and conductor G, and forms a somewhat circular path along a plane perpendicular to each of the east, west, north and south planes and near the top of radiation tip 302. The six radiation coils 325-350 may conveniently be described as disposed within the planes of a six-sided cube-like shape. Although not shown, the radiation tip 302 is housed in an electrically transparent chamber that protects the tip 302 from damage but does not affect the electromagnetic field pattern emanating therefrom. The diameter of the substantially circular paths of each of the coils 325-350 is preferably about the same as the diameter of the probe 300, or about 3-10 millimeters, although alternative diameters are also possible. In fact, the diameter can be several centimeters in diameter.

To shield the coaxial nesting conductors 310 from one another, isolating material 320 is preferably placed between certain of the nesting conductors 310. In some cases, the isolating material may be ceramic. As shown, isolating material 320 is placed between conductors A and B, conductors B and C, conductors C and D, conductors D and E and conductors E and F.

To keep the conduction member 301 cool, a coolant (such as water, gas or other thermo-conductive fluid 315) is preferably circulated inbound through the region between the first pair of conductors 310, namely, between conductors G and A, and outbound through the region within the innermost conductor 310, namely, within conductor F. However, one skilled in the art will recognize that coolant can circulate through other paths within probe 300. The pressure of the coolant may be maintained below, at or above atmospheric pressure, for example, at approximately 10 Atm. Although not shown, the distal end of the conduction member 301 is preferably capped by a substantially electrically opaque and coolant-tight shield. That way, the electromagnetic fields caused by the conductors 310 are shielded from the distal end, and substantially no coolant being circulated can enter and cool the radiation tip 302. The radiation coils 325, 330, 335, 340, 345 and 350 pass through the shield to the conductors 310.

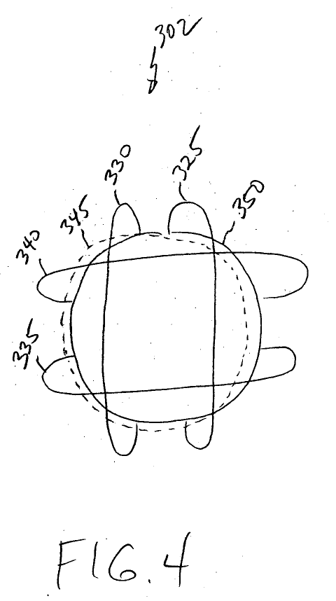

FIG. 4 is a top view of the radiation tip 302 of the probe 300 in accordance with an embodiment of the present invention. The radiation tip 302 illustrates the position of the six radiation coils 330, 335, 340, 345, and 350. Namely, radiation tip 302 includes radiation coil 325 in the east plane, radiation coil 330 in the west plane, radiation coil 335 in the south plane, radiation coil 340 in the north plane, radiation coil 345 in the lower plane near the distal end of the conduction member 301, and radiation coil 350 in the upper plane away from the distal end of the conduction member 301.

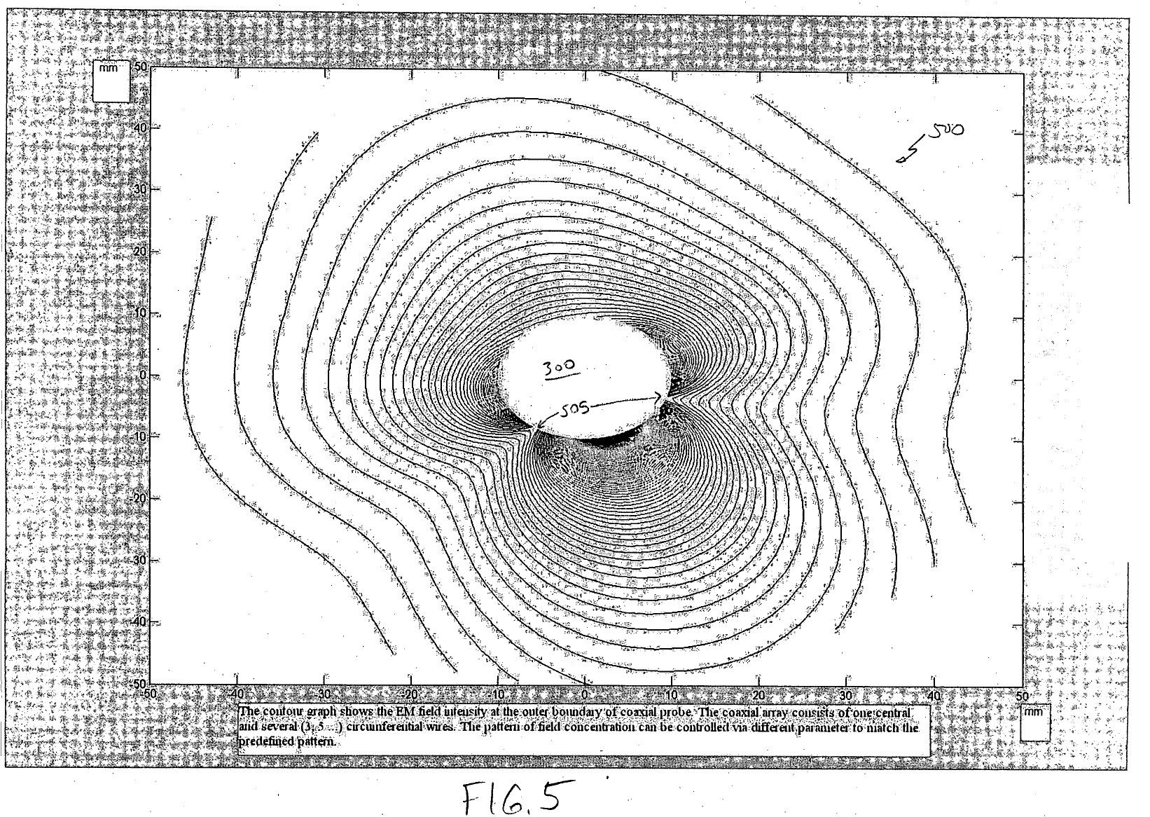

FIG. 5 is a contour graph showing a first example electromagnetic field intensity pattern 500 at the outer boundary of and in a plane perpendicular to the probe 300. The pattern 500 is generated by conducting two current signals (tones) to generate two interferential electromagnetic fields. As shown, the probe 300 has a 20 millimeter diameter. (Although seemingly elliptical in shape, the coordinate spacing on the x and y axes are not identical.) Because of the interference between the two electromagnetic fields caused by two electric current signals applied to two different radiation coils of coils 325-350, the interference pattern 500 is not merely circular. In fact, two near zero electromagnetic field regions 505 are shown. This near zero electromagnetic field regions 505 may be desirable, for example, if a healthy vessel is near the target ablation zone. The healthy vessel can be placed within one of the cancellation regions to reduce its risk of damage.

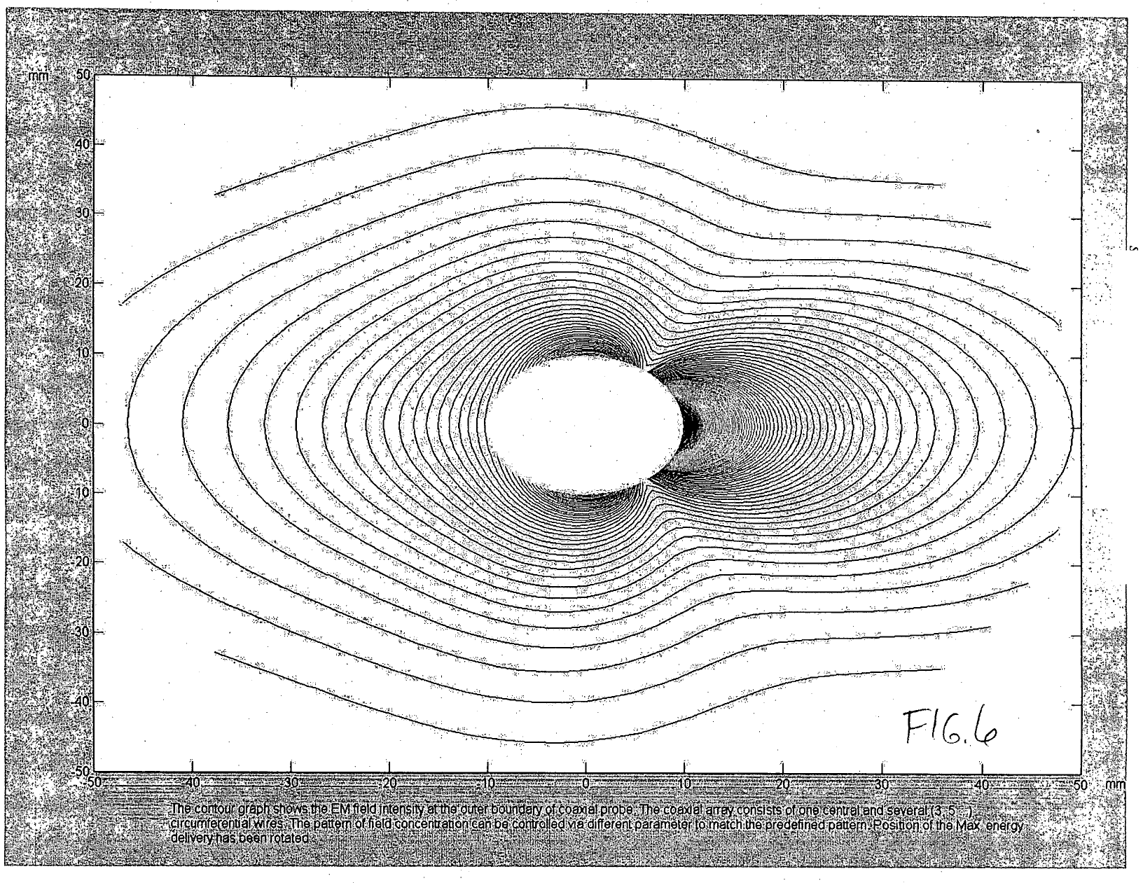

FIG. 6 is a second contour graph showing a second example electromagnetic field intensity pattern 600 at the outer boundary of the probe 300. The pattern 600 is also generated by conducting two current signals to generate two interfering electromagnetic fields. As can be seen, the radiation pattern 600 is substantially the same as the pattern 500 illustrated in FIG. 5, except that it has been rotated about 90 degrees. It will be appreciated that the electromagnetic field pattern 600 was achieved by maintaining the probe 300 in a stationary position, and transmitting the current signals along two different conductors 310 to two different radiation coils of coils 325-350. This may be advantageous when multiple electromagnetic field applications are desirable but repositioning of the probe 300 is not.

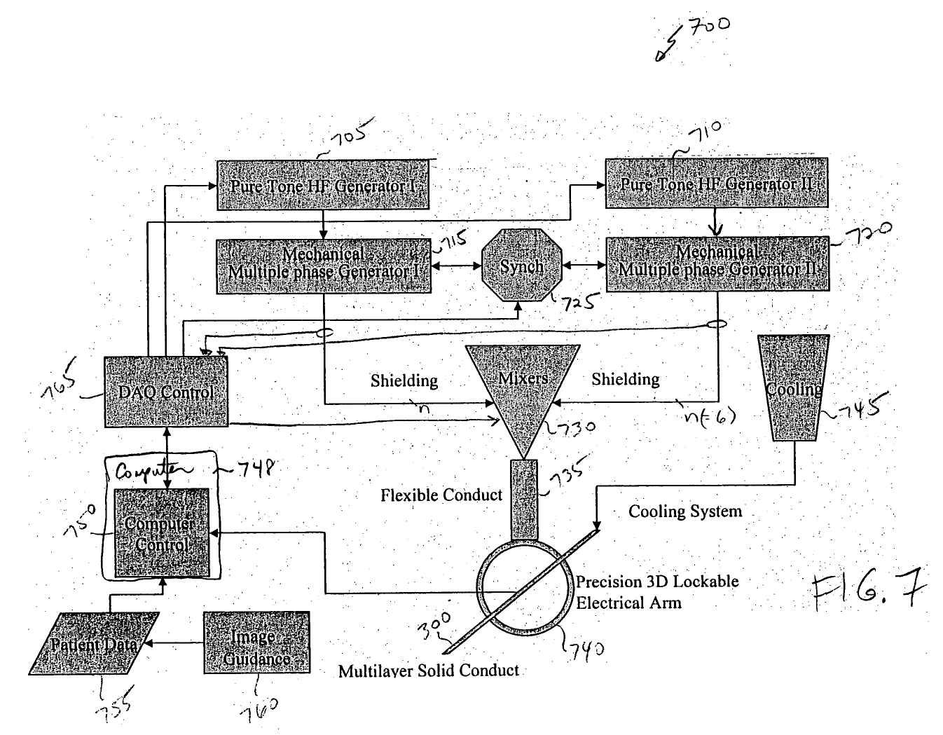

FIG. 7 is a block diagram illustrating an interferential electromagnetic field generation system 700 in accordance with an embodiment of the present invention. System 700 includes two pure-tone high-frequency generators 705 and 710. However, although system 700 is shown as having two high-frequency generators 705 and 710, the system 700 can be implemented with any number of high-frequency generators 705 and 710. Further, the high-frequency generators 715 and 710 may be within in a single unit. High-frequency generator 705 generates a first wave (wave A). High-frequency generator 710 generates a second wave (wave B). Wave A and wave B may be the same frequency or different frequencies, each preferably being in the range of 1 to 30 GHz. However, one of wave A or wave B is preferably a multiple of the other, with zero angle phase difference. When there are more than two high-frequency generators, each wave is preferably a multiple of the slowest wave (e.g., 1, 2, 3 and 4; or 1, 2, 4 and 8; or 1, 2, 3, 7; or 1, 1, 2, 5) with zero angle phase difference.

Each of the high-frequency generators 705 and 710 is coupled to a respective multiple phase generator 715 and 720. Alternatively, the multiple phase generators 715 and 720 can be part of a single unit multiple phase generator or can be part of the high-frequency generators 705 and 710 in single or multiple units. Multiple phase generator 715 receives wave A from high-frequency generator 705 and generates multiple output signals, each output signal having the same frequency as wave A with phase adjustment. The output signals of the multiple phase generator 715 need not be equally spaced apart in phase. The number of output signals from the multiple phase generator 715 is preferably equal to the number of radiation coils 325-350 in probe 300. Similarly, multiple phase generator 720 receives wave B from high-frequency generator 710 and generates multiple output signals, each output signal having the same frequency as wave B with phase adjustment. The output signals of multiple phase generator 720 need not equally spaced part in phase. The number of output signals from the multiple phase generator 720 is also preferably equal to the number of radiation coils 325-350 in probe 300. The output signals generated by the multiple phase generators 715 and 720 are sent to mixers 730.

Mixers 730 adjust the intensity of each of the output signals and mix the intensity- adjusted waves together onto each of the conductors 310 (FIG. 3). The intensity can be zero, an integral multiple (1, 2, 4, etc.) or a non-integral multiple (0.3, 1.4, etc.). A synchronization module 725 is coupled between the multiple phase generators 715 and 720 to control the phase offsets of multiple phase generators 715 and 720. Although mixers 730 are being described as adjusting the intensity of the waves, namely, of waves A and B, one skilled in the art will recognize that the multiple phase generators 715 and 720 or the synchronization module 725 can adjust the intensity of the output signals. If the synchronization module 725 adjusts the intensity of the output signals, the synchronization module 725 will receive control information for controlling the intensity vectors. The synchronization module 725 can be part of the multiple phase generators 715 and 720.

Mixers 730 are coupled to a flexible conduct 735. Flexible conduct 735 is connected to a precision 3D lockable electrical arm 740, which holds the probe 300 (identified as a multilayer solid conduct). The precision 3D lockable electrical arm 740 enables a physician to manipulate and lock the direction, geographic position, etc. of the probe 300 relative to the target. The patient is typically strapped in place. The probe 300 is typically placed so that the radiation tip 302 is disposed at a desirable position relative to the tumor 205 for application of the electromagnetic field pattern. The flexible conduct 735 is allowed to move so that movement of the precision 3D lockable electrical arm 740 does not disrupt the electrical flow of the mixed output signals from the mixers 730 to the probe 300. The length of the wire from the mixers 730 through the flexible conduct to the radiation tip 302 of the probe 300 and back is preferably selected to be about a round multiple of half the wavelength of the slowest frequency of the wave. For example, the wavelength of a 10 GHz wave is about 1 inch. Accordingly, the length of the wire should be a multiple of 0.5 inch.

A cooling system 745 is coupled to the probe 300 and circulates coolant in a manner as described above with regard to FIGS. 3AB, 3CD and 3EF. As shown, the cooling system 745 applies coolant to a proximal end of the probe 300 and circulates it through the conduction member 301 of the probe 300, thereby keeping the conduction member 301 cool and the radiation tip 302 hot.

A computer control 750 operating on a computer system 748 receives probe input (e.g., probe angle, tip position, radiation coil location relative to the tumor 205, temperature, etc.) from probe 300 (in this example). Although the probe input is shown as coming from probe 300, the probe input can come from any device or sensor. The computer control 750 also receives patient data 755 (e.g., tumor shape, position, size, etc.) (in this example generated by image guidance system 760). Although the patient data 755 is shown as being received from image guidance system 760, the patient data 755 can come from any source, e.g., from 2D image capture, 3D image capture, 4D image capture, drawn by the physician based on visual inspection, ultrasound, x-ray, MRI, etc.

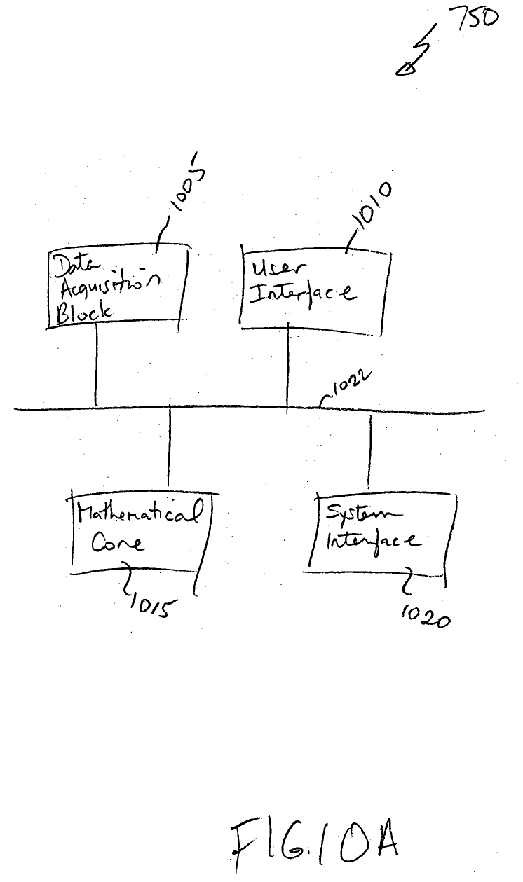

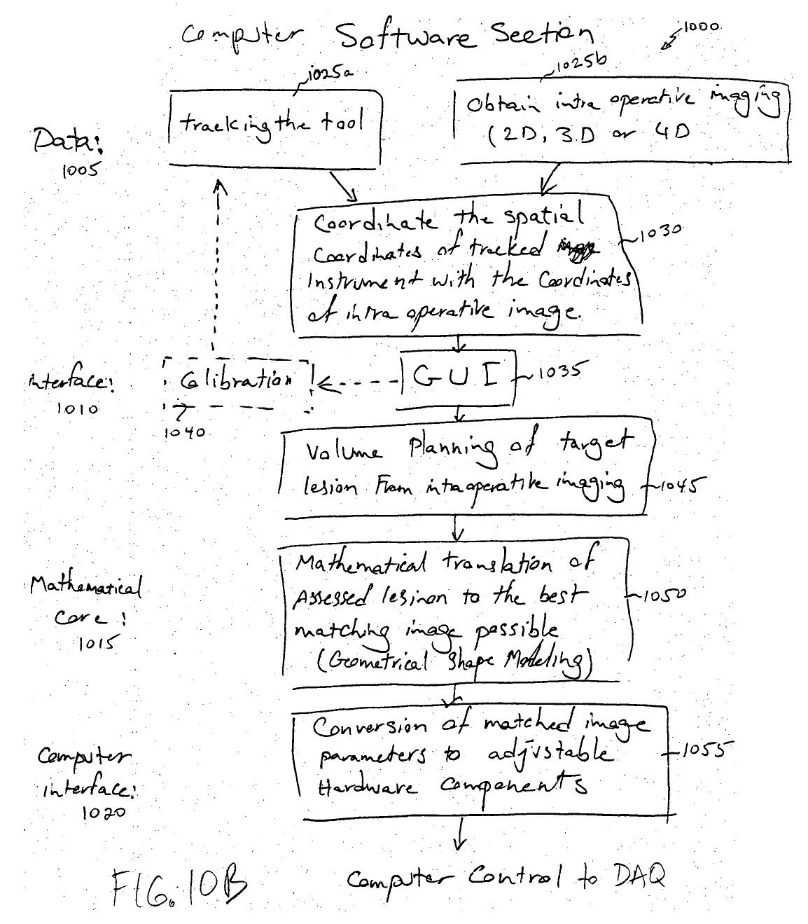

Based on the probe input and patient data 755, the computer control 750 generates and sends system control information to a DAQ (Data Acquisition) control 765. Control information may include the best frequencies, phases, intensities, etc. to generate a desired electromagnetic field pattern based on the patient data 755 and probe input. DAQ control 765 receives the control information from the computer control 750, and possibly senses current amounts from the output of the multiple phase generators 715 and 720 (as an alternative or addition to temperature sensing of the radiation tip 302). In response, the DAQ control 765 sends high-frequency generator control information (e.g., frequency control data, etc.) to the high-frequency generators 705 and 710, sends synchronization module control information (e.g., phase control data, intensity control data, and/or the like.) to the synchronization module 725, and sends mixers control information (e.g., intensity control data, mixing control data, and/or the like) to the mixers 730. The mathematical models for computing the frequency, phase, intensity, etc. for generating the desired electromagnetic field pattern are discussed in greater detail below with reference to FIGS. 10A and 10B. Although the DAQ control 765 and computer 748 are shown as separate units, these components can be part of a single unit.

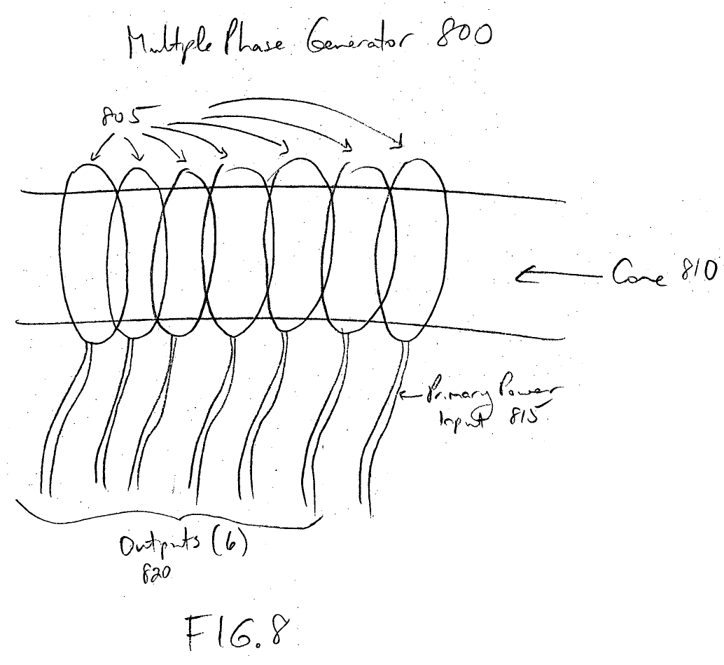

FIG. 8 is a diagram illustrating details of a multiple phase generator 800 embodiment which can be used in the interferential electromagnetic field generation system 700 of FIG. 7. Although prior art multiple phase generators can alternatively be used in system 700, servo-controlled mechanical multiple phase generator 800 offers additional benefits. Multiple phase generator 800 includes multiple servo-controlled rotary pickups 805 positioned about a ferromagnetic core 810. Each of the pickups 805 comprises a controlled wire length to assure no phase change based on the wire length. Pickups 805 include a primary input 815 and multiple outputs 820. Each of the outputs 820 is preferably rotatable 360 degrees about the core 810. Although the primary input 815 may also be rotatable about the core 810, it is preferably in a fixed position. In the example shown, the pickups 805 include six outputs 820, one for each conductor 310. The primary input 815 supplies a pure-tone frequency wave to the core 810. Based on the rotational position of the signal outputs 820 relative to the primary input 815, the outputs 820 through electromagnetic inductance modify the phase angle of the supplied wave relative to the primary input 815 while maintaining its frequency. Although not shown, the primary input 815 may be in the center position. This generator 800 enables a servo controller (not shown) to adjust the six phase output signals 820 to any phase angle relative to a primary power input 815.

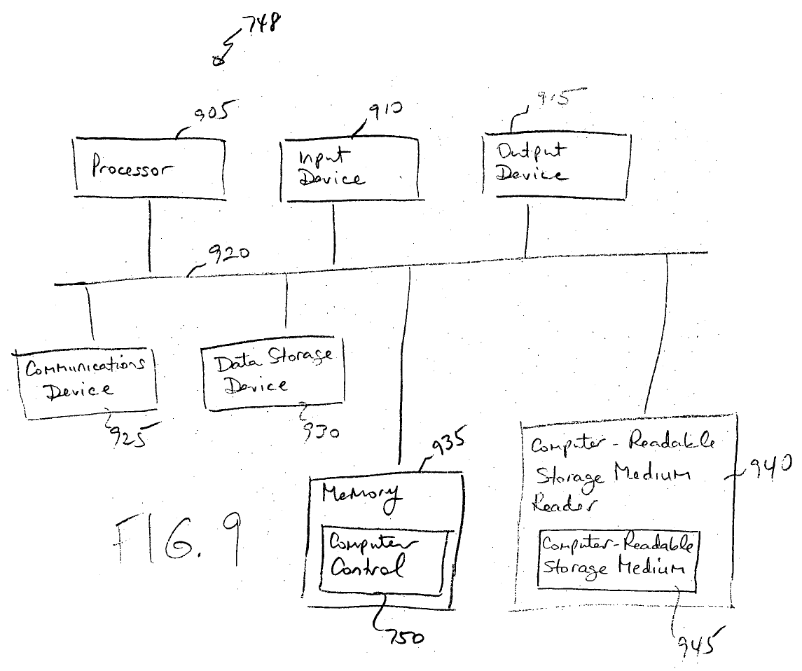

FIG. 9 is a block diagram illustrating details of computer system 748 in accordance with an embodiment of the present invention. The computer system 748 includes a processor 905, such as an Intel Pentium® microprocessor or a Motorola Power PC® microprocessor, coupled to a communications channel 920. The computer system 748 further includes an input device 910 such as a keyboard or mouse, an output device 915 such as a cathode ray tube display, a communications device 925, a data storage device 930 such as a magnetic disk, and memory 935 such as Random-Access Memory (RAM), each coupled to the communications channel 920. Memory 935 stores computer control 750, which is described in greater detail with reference to FIGS. 7, 10A and 10B. One skilled in the art will recognize that, although the data storage device 930 and memory 935 are illustrated as different units, the data storage device 930 and memory 935 can be parts of the same unit, distributed units, virtual memory, etc. The communications device 925 may be coupled to a network such as the wide-area network commonly referred to as the Internet.

The data storage device 930 and/or memory 935 may store an operating system such as the Microsoft Windows NT or Windows/95 Operating System (OS), the IBM OS/2 operating system, the MAC OS, or UNIX operating system and/or other programs. It will be appreciated that a preferred embodiment may also be implemented on platforms and operating systems other than those mentioned. An embodiment may be written using JAVA, C, and/or C++ language, or other programming languages, along with an object oriented programming methodology.

One skilled in the art will recognize that the computer system 748 may also include additional information, such as additional network connections, additional memory, additional processors, LANs, input/output lines for transferring information across a hardware channel, the Internet or an intranet, etc. One skilled in the art will also recognize that the programs and data may be received by and stored in the system in alternative ways. For example, a computer-readable storage medium (CRSM) reader 940 such as a magnetic disk drive, hard disk drive, magneto-optical reader, CPU, etc. may be coupled to the communications bus 920 for reading a computer-readable storage medium (CRSM) 945 such as a magnetic disk, a hard disk, a magneto-optical disk, RAM, etc. Accordingly, the computer system 748 may receive programs and/or data via the CRSM reader 940. Further, the term “memory” herein is intended to cover all data storage media whether permanent or temporary.

FIG. 10A is a block diagram illustrating details of computer control 750. Computer control 750 includes a data acquisition block 1005, a user interface 1010, a mathematical core 1015 and a system interface 1020, each able to communicate over a communications channel 1022.

The data acquisition block 1005 obtains patient data 755 from image guidance 760. Image guidance 760 may generate the patient data 755 using 2D, 3D or 4D (ultrasound) operative imaging. Alternatively, the patient data 755 may be generated by the physician or supplied by some other device or person. Patient data 755 may include tumor shape, position, size, etc. The data acquisition block 1005 also obtains probe input from sensors (not shown) on the probe 300, on the precision lockable electrical arm 740 or on some external mechanism. Probe input may include probe angle, tip position, coil location relative to the tumor 205, temperature, etc.

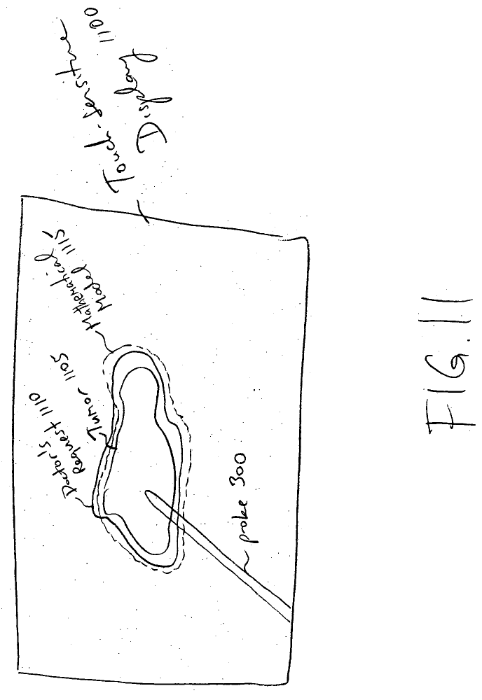

Operating with the output device 915, the user interface 1010 presents the patient data 755 preferably as a 3D graphical image. FIG. 11 illustrates a 2D (since 3D would be too difficult to draw) graphical image of the tumor 205 as 2D tumor image 1105. Operating with the output device 915, the user interface 1010 displays an image of the probe 300 relative to the tumor 205.

Operating with the input device 910 and the output device 915, the user interface 1010 may enable the physician/user to input, e.g., outline, a desired electric field (or temperature or the like) pattern based on the patient data 755. For example, the physician may outline around the tumor 1105, attempting to minimize the electric field near critical vessels and/or organs. Alternatively, the mathematical core 1015 (described below) may generate the computer's best guess of an electric field pattern to be applied to the tumor 1105 based on the relevant variables. In this embodiment, the computer's best guess will be substantially equivalent to the dimensions of the tumor 1105. In another embodiment, the computer's best guess will demand a cell-destroying temperature (e.g., 70 degrees Celsius) within the tumor 1105 (the tumor zone), a higher cell-destroying temperature (e.g., 73 degrees Celsius) in the margin outside the tumor 205 (the margin zone) to assure margin enhancement and that no tumoral residue remains, and safe temperature (e.g., 37 degree Celsius) outside that margin zone (the normal zone). The user interface 1010 may enable the physician to adjust/modify the computer's best guess to add his common sense, experience and skills to the pattern selected. FIG. 11 illustrates the physician's request 1110 (whether generated by the computer, edited by the physician, or completely generated by the physician). Operating with the output device 915, the user interface 1010 preferably displays the mathematical model of the electric field to be generate by the system 700.

The mathematical core 1015 uses the physician's request 1110, probe input and the limitations of the system 700 to determine the best possible electric field pattern. For example, the size and shape of each radiation coils 325-350, the position and number of radiation coils 325-350, the number of high-frequency signals combinable over each conductor 310, etc. may limit the electric field patterns available. Based on these limitations, the closest available mathematical model is generated. Alternatively, the mathematical core 1015 may generate closest possible electric field pattern options, which may be presented to the physician as options. The physician can then select from the options. FIG. 11 illustrates the closest possible electric field pattern as mathematical model 1115. As described below, if the physician is not pleased with the mathematical model 1115 (or mathematical model options), the physician may adjust parameters, e.g., the position of the probe 300 or the desired electric field pattern (i.e., the physician's request 1110).

In one embodiment, the mathematical core 1015 generates the best electric field pattern relative to the physician's request 1110 according to the following algorithm:
1. Generate an array of current variables (e.g., frequency, phase and intensity) for each of the radiation coils 325-350. An example array will likely include all possible combinations of frequency, phase and intensity for the radiation coils 325-350 assuming a predetermined step amount between each value of each variable. The step amount may differ based on the variable. For example, the array may account for frequencies from 1 GHz to 30 GHz in step amounts of 1 GHz. The array may account for phases from 0 to 360 degrees in step amounts of 10 degrees. The array may account for intensities in integral multiples from 1 to 5 in step amounts of 1.
2. Use Biot-Savart law

$${dB}^{⟶} = \frac{{μ}_{0} I}{4 π} \frac{dL \times ( r - {r}^{′} )}{{\vert r - {r}^{′} \vert}^{3}}$$

to convert each current to a magnetic field from the corresponding radiation coil 325-350. Using Biot-Savart law, the mathematical core 1015 can compute the radiation from the corresponding radiation coil 325-350 at a predetermined set of points out to a predetermined distance from the tumor 1105. For example, the predetermined set of points may be every two millimeters in three-dimensional space, out to 10 centimeters away from the periphery of the tumor 1105. The magnetic field may be computed for each radiation coil 325-350 and applying vectorial superposition to generate the magnetic field pattern emanating from all radiation coils 325-350 of the radiation tip 302.
3. Use Maxwell Equations to convert the magnetic field pattern to an electric field pattern based on tissue electrical characteristics. For example, the electrical characteristics of tissue have been determined based on the type of tissue, e.g., liver tissue, liver tumor tissue, breast tissue, breast tumor tissue, etc.
4. Use known thermal and electrical characteristics of tissue to convert the electric field pattern to a heat distribution pattern.
5. Use base temperature, the thermal characteristics and geographic positions of the various tissues in the field, and application time to generate an expected temperature pattern.
6. Use weighting methods to compare the expected temperature pattern against the desired temperature pattern to determine the best options available. The best options can be determined by comparing point by point the percentage deviation of the expected temperature pattern from the desired temperature pattern (whether this percentage is computed for the pattern in the entire field and/or separately for each zone). The weighing method may be based on criteria to facilitate the analysis of energy distribution patterns. Absolute requirements may enable the mathematical core 1015 to ignore certain steps to speed up calculations. For example, if a certain phase and amplitude leads to a temperature below some minimum threshold in the margin zone (e.g., 42 degrees Celsius), the mathematical core 1015 may ignore this phase and amplitude option. Weighting can also be applied to each of the zones (e.g., the tumor zone, the margin zone and the normal zone) to generate a value representing how well the temperature pattern matches the desired pattern.

Based on the parameters generated by the mathematical core 1015, the system interface 1020 generates system control information to the DAQ Control 765. The system control information may include the best frequencies, phases, intensities, etc. to generate the electric field pattern of the mathematical model 1115.

FIG. 10B is a flowchart illustrating a method 1000 of controlling the components of the computer control 750 to generate an electric field pattern based on a target pattern. For convenience, the method 1000 will be shown relative to the image in FIG. 11. Method 1000 begins with the data acquisition block 1005 in step 1025a obtaining intra-operative imaging information (patient data 755) of the tumor 205 whether using 2D, 3D or 4D (ultrasound) models. In step 1025b, the data acquisition block 1005 receives probe input (e.g., probe angle, tip position, coil location relative to the tumor 205, temperature, etc.) from sensors (not shown). The data acquisition block 1005 in step 1030 computes the spatial coordinates for display on display 915. The user interface 1010 in step 1035 presents the tumor 1105 and the probe 300 on a graphical user interface, so that the physician can witness the position of the probe 300 relative to the tumor 1105. If the position of the probe 300 relative to the tumor 1105 is not ideal, as determined by the physician, the position of the probe 300 can be adjusted. Method 1000 will return to step 1025a, to make adjustments of the spatial coordinates and display.

If the physician believes that the position of the probe 300 relative to the tumor 1105 is “ideal”, then in step 1040 the physician can use the user interface 1010 to design (e.g., outline) the desired electric field pattern (i.e., the physician's request 1110). The mathematical core 1015 in step 1045 applies volume and wave analysis to generate the closest interferential field pattern relative to the physician's design. The closest interferential electric field pattern is illustrated as the mathematical model 1115.

The computer interface 1010 displays the mathematical model 1115. If the physician is not pleased with the result, the physician can adjust the position of the probe 300 (at such time the method 1000 will return to step 1025a) or can redraw the desired pattern (at which time the method 1000 will return to step 1045). It will be appreciated that the physician can make adjustments multiple times. The treatment design selected may include radiating different portions of the tumor 1105 separately from the same probe position or from different probe positions.

As soon as the physician is pleased with the selected treatment plan, the computer-to-mechanical system interface in step 1050 can convert the mathematical pattern to parameters for the hardware components of system 700 (e.g., for the high-frequency generators 705 and 710, for the synchronization module 725, for the multiple phase generators 715 and 720, for the mixers 730, etc.). Method 1000 then ends.

FIG. 11 is a diagram illustrating the user interface 1010 for receiving physician input and representing diagrammatically the radiation pattern to be generated by the interferential electric field generation system 700. The user interface 1010 may be applied on a touch-sensitive display 1100. As shown, the user interface 1010 displays the tumor 1105, the probe 300, the physician's requested pattern 1110 (whether generated by the physician from scratch, or by the system 700 and edited by the physician), and the mathematical model 1115 of the best-possible electric field pattern possible.

The foregoing description of the preferred embodiments of the present invention is by way of example only, and other variations and modifications of the above-described embodiments and methods are possible in light of the foregoing teaching. For example, although the probe 300 is illustrated as positioned within the tumor 205, one skilled in the art will realize that, because of wave cancellations, the probe 300 may be positioned near or away from the tumor 205. The various embodiments set forth herein may be implemented utilizing hardware, software, or any desired combination thereof. For that matter, any type of logic may be utilized which is capable of implementing the various functionality set forth herein. Components may be implemented using a programmed general purpose digital computer, using application specific integrated circuits, or using a network of interconnected conventional components and circuits. Connections may be wired, wireless, modem, etc. The embodiments described herein are not intended to be exhaustive or limiting. The present invention is limited only by the following claims.

1. A probe for generating an electromagnetic field, comprising:
a first conductor for receiving a first current signal;
a second conductor for receiving a second current signal;
a first radiation coil coupled to the first conductor for radiating a first electromagnetic field based on the first current signal; and
a second radiation coil coupled to the second conductor for radiating a second electromagnetic field based on the second current signal, the first and second electromagnetic fields causing an interferential electromagnetic field pattern.

2. The probe of claim 1, wherein the first conductor is a coaxial conductor.

3. The probe of claim 1, wherein the first conductor and the second conductor are nesting coaxial conductors.

4. The probe of claim 1, wherein the first radiation coil is coupled between the first conductor and ground, and the second radiation coil is coupled between the second conductor and ground.

5. The probe of claim 1, wherein the first radiation coil and the second radiation coil are in parallel planes.

6. The probe of claim 1, wherein the first radiation coil and the second radiation coil are in perpendicular planes.

7. The probe of claim 1, wherein the probe is less than 10 millimeters in diameter.

8. The probe of claim 1, wherein the first current signal has a first frequency, and the second current signal has a second frequency which is a substantially perfect multiple of the first frequency.

9. The probe of claim 1, wherein the first current signal and the second current signal are out of phase.

10. The probe of claim 1, wherein the first current signal and the second current signal are in phase.

11. The probe of claim 1, wherein the first current signal comprises an interferential current signal of at least two currents.

12. A method for generating an electromagnetic field, comprising:
receiving a first current signal;
receiving a second current signal;
radiating a first electromagnetic field based on the first current signal; and
radiating a second electromagnetic field based on the second current signal, the first and second electromagnetic fields causing an interferential electromagnetic field pattern.

13. The method of claim 12, wherein the first current signal has a first frequency, and the second current signal has a second frequency which is a substantially perfect multiple of the first frequency.

14. The method of claim 12, wherein the first current signal and the second current signal are out of phase.

15. The method of claim 12, wherein the first current signal and the second current signal are in phase.

16. The method of claim 12, wherein the first current signal comprises an interferential current signal of at least two currents.

17. A system for generating an electromagnetic field, comprising:
means for receiving a first current signal;
means for receiving a second current signal;
means for radiating a first electromagnetic field based on the first current signal; and
means for radiating a second electromagnetic field based on the second current signal, the first and second electromagnetic fields causing an interferential electromagnetic field pattern.

18. A probe for generating an electromagnetic field, comprising:
a conduction member for conducting at least two current signals; and
a radiation tip coupled to the conduction member for radiating an electromagnetic field based on the at least two current signals.

19. The probe of claim 18, wherein the conduction member includes six nesting coaxial conductors, each for conducting a current signal.

20. The probe of claim 19, wherein the radiation tip includes six radiation coils, each radiation coil has one end coupled to a respective one of the six coaxial conductors and the other end coupled to ground.

21. The probe of claim 20, wherein the conduction member includes a seventh nesting coaxial conductor that is coupled to ground and to the grounded end of each radiation coil.

22. The probe of claim 21, wherein the six radiation coils are positioned in different planes.

23. The probe of claim 21, wherein the six radiation coils are positioned substantially in the planes of a cube.

24. The probe of claim 19, wherein isolating material is placed between several of the nesting coaxial conductors.

25. The probe of claim 19, wherein a coolant circulates between two adjacent nesting conductors.

26. The probe of claim 25, wherein the two adjacent nesting conductors include the two outermost conductors.

27. The probe of claim 19, wherein a coolant circulates within one of the conductors.

28. The probe of claim 18, wherein the at least two current signals are conducted on a single conductor as an interferential current signal, and the radiation tip includes one radiation coil coupled to this single conductor.

29. The probe of claim 18, wherein the at least two current signals are conducted on different conductors.

30. A method for generating an electromagnetic field, comprising:
conducting at least two current signals; and
radiating an electromagnetic field based on the at least two current signals.

31. The method of claim 30, wherein the at least two current signals are conducted on a single conductor as an interferential current signal, and the electromagnetic field is radiated from one radiation coil coupled to this single conductor.

32. The method of claim 30, wherein the at least two current signals are conducted on different conductors.

33. A system for generating an electromagnetic field, comprising:
means for conducting at least two current signals; and
means for radiating an electromagnetic field based on the at least two current signals.

34. A radiation tip for generating an electromagnetic field pattern to ablate tissue, comprising:
a first radiation coil for radiating a first electromagnetic field based on a first current signal; and
a second radiation coil for radiating a second electromagnetic field based on a second current signal, the first electromagnetic field and the second electromagnetic field causing an interferential electromagnetic field pattern for ablating tissue.

35. The probe of claim 34, wherein the first radiation coil and the second radiation coil are in parallel planes.

36. The probe of claim 34, wherein the first radiation coil and the second radiation coil are in perpendicular planes.

37. The probe of claim 34, wherein the radiation tip is less than 10 millimeters in diameter.

38. The probe of claim 34, wherein the first current signal has a first frequency, and the second current signal has a second frequency which is a substantially perfect multiple of the first frequency.

39. The probe of claim 34, wherein the first current signal and the second current signal are out of phase.

40. The probe of claim 34, wherein the first current signal and the second current signal are in phase.

41. The probe of claim 34, wherein the first current signal comprises an interferential current signal of at least two currents.

42. A method for generating an electromagnetic field pattern to ablate tissue, comprising:
radiating a first electromagnetic field based on a first current signal; and
radiating a second electromagnetic field based on a second current signal, the first electromagnetic field and the second electromagnetic field causing an interferential electromagnetic field pattern for ablating tissue.

43. The method of claim 42, wherein the first current signal has a first frequency, and the second current signal has a second frequency which is a substantially perfect multiple of the first frequency.

44. The method of claim 42, wherein the first current signal and the second current signal are out of phase.

45. The method of claim 42, wherein the first current signal and the second current signal are in phase.

46. The method of claim 42, wherein the first current signal comprises an interferential current signal of at least two currents.

47. A system for generating an electromagnetic field pattern to ablate tissue, comprising:
means for radiating a first electromagnetic field based on a first current signal; and
means for radiating a second electromagnetic field based on a second current signal, the first electromagnetic field and the second electromagnetic field causing an interferential electromagnetic field pattern for ablating tissue.
